(19) United States
(12) Patent Application Publication
(10) Pub. No.: US 2010/0114151 A1
(43) Pub. Date: May 6, 2010

(54) METHODS AND DEVICES FOR DEPLOYMENT INTO A LUMEN

(76) Inventors: James R. Mujwid, Crystal, MN (US); William S. Tremulis, Redwood City, CA (US); Karl A. Jagger, Minnetonka, MN (US); John R. Frigstad, St. Anthony, MN (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/607,046

(22) Filed: Oct. 27, 2009

Related U.S. Application Data
(60) Provisional application No. 61/108,598, filed on Oct. 27, 2008.

(51) Int. Cl. A61M 29/00; A61M 25/00

(52) U.S. Cl. 606/194; 604/266

(57) ABSTRACT

The present invention is directed to an intracorporeal occlusion device having denuding elements, motion transfer converter or anti-migration features or a combination of one or more of these features. The disclosed occlusion device can denude the body lumen through a rotational motion of the denuding elements within a lumen. The delivery system can transfer a linear motion of a handle to a combination of linear and rotational movements of the occlusion device for placement and denuding action. Anti-migration features provide free spinning of the main body of the occlusion device to prevent migration of the deployed occlusion device, especially with rotatable denuding elements.

This application claims the benefit of U.S. provisional patent application No. 61/108,598, filed Oct. 27, 2008 and hereby incorporates by reference the entire content of that provisional application into this application.

### BACKGROUND

Conventional contraceptive strategies generally fall within three categories: physical barriers, drugs and surgery. While each have certain advantages, they also suffer from various drawbacks. Barriers such as condoms and diaphragms are subject to failure due to breakage, displacement and misplacement. Drug strategies, such as the pill and Norplant™, which rely on artificially controlling hormone levels, suffer from known and unknown side-effects from prolonged use. Surgical procedures, such as tubal ligation and vasectomy, are very effective, but involve the costs and attendant risks of surgery, and are frequently not reversible.

There exist various situations in which it is desirable to implant embolic or occlusive devices within lumens or anatomical passageways within the bodies of human or animal subjects.

For example, it has been known to implant occlusive devices into the fallopian tubes of females or the vas deferens of males for contraceptive purposes. Examples of implantable occlusive devices useable for such purposes are described in U.S. Pat. No. 6,176,240 (Nikolchev, et al.) and U.S. Pat. No. 6,705,323 (Nikolchev, et al.) and U.S. Pat. No. 7,506,650 (Lowe, et al.) and U.S. Pat. No. 6,096,052 (Callister et al.) entitled Occluding Device and Method of Use and U.S. Pat. No. 6,432,116 (Callister et al.) entitled Occluding Device and Method of Use, the entireties of all such United States patents being expressly incorporated herein by reference. Some of these devices have been constructed and/or implanted in a manner to facilitate tissue ingrowth subsequent to implantation of the device such that, after such tissue ingrowth has occurred, the ingrown tissue alone or in combination with the implanted device will provide complete occlusion of the lumen of the fallopian tube or vas deferens. The Essure device from Conceptus is an example of such device, and it can also cause some denuding of the fallopian tube when a coil of the Essure device is deployed within the fallopian tube.

The above incorporated U.S. patent application Ser. No. 08/770,123 (Callister et al.) described various embodiments of lumen occluding devices that may be used to occlude the lumen of a fallopian tube or vas deferens, some of which may deliver a drug, such as a contraceptive agent.

The fallopian tubes tend to exude objects through peristaltic (muscular contraction) and ciliated forces, and the direction of the force varies with menstrual cycle. These forces may result in the displacement of an implant. Past implant designs have been designed to counteract these forces, examples of which are found in U.S. patent application Ser. No. 10/880,355, and Ser. No. 10/746,131, which are hereby incorporated by reference. However these designs may result in the puncture of the fallopian tube wall because they have unprotected free ends. Therefore it is desirable to incorporate a design which counteracts the forces in the fallopian tube while not completely puncturing the tube walls.

### SUMMARY OF THE DESCRIPTION

The present invention is directed to occlusion devices, delivery systems for such devices and methods of using such devices and systems for occluding body passageways, particularly reproductive body lumens such as a female's fallopian tubes and a male's vas deferens.

This invention generally relates to the field of occluding devices, delivery systems for such devices and methods of using such devices and systems in the occlusion of body passageways. The invention is particularly useful for occluding reproductive lumens such as a female patient's fallopian tubes or a male patient's vas deferens to effect contraception. Although the occlusion of a patient's reproductive lumens will be discussed herein in detail, it can be appreciated that the devices, methods and systems described herein can easily be adapted to occlude a patient's arteries or veins in a variety of situations. Those skilled in the art will immediately recognize that various combinations, modifications, and equivalents of the inventions described herein can be used without departing from the scope of these inventions.

In an embodiment, the present invention discloses an expandable sterilization occlusion device with a denuding feature, and can further include a rotational mechanism to actuate a denuding element when in the body lumen for stimulating the body lumen to encourage tissue growth. In an aspect, a delivery system is inserted into a body lumen, where the occlusion device is first partially released, and where an expansive denuding element of the occlusion device is partially or fully expanded. After the expansion of the denuding element, the occlusion device is rotated a predetermined amount of rotation, allowing the expanded denuding element to denude the body lumen. The occlusion device is preferably still attached to the delivery system to allow the handling of the delivery system to act on the movement of the occlusion device. After the rotational action, the occlusion device is then released from the delivery system and deployed into the body lumen, and the delivery system is retracted from the body lumen.

The occlusion device embodying features of the invention can have at least an expansive denuding element, preferably self-expanding and secured by at least one end thereof to a central location within the device. The denuding element can be expanded when being fully or partially released to the body lumen. When fully released or partially released, expanded or partially expanded, the occlusion device can be rotated, preferably a full rotation of 360 degrees, but can be any desired rotational angle.

In an embodiment, the occlusion device is still attached to a delivery system at the beginning of a rotational action. For example, the occlusion device can be first partially released from a delivery system (such as a catheter) by unsheathing a distal portion of the occlusion device to expose an expansive denuding element. In this partially released configuration, the expansive denuding element can be fully expanded, or partially expanded. The occlusion device is then released after the rotational action, for example, by performing a removal process to completely release the occlusion device, or the occlusion device is already released at the completion of the rotational action (e.g. the rotational action unscrews the occlusion device from a wire in the delivery system).

The occluding device may be delivered to an intracorporeal location through a delivery system which has a delivery catheter with an inner lumen configured to receive the occluding device in a constricted configuration, where the expansive denuding elements (together with other expansive elements) of the occluding device are compressed or radically restrained within the delivery catheter before being released from the delivery catheter. A pusher element can be, in one embodiment, slidably disposed within the inner lumen of the delivery catheter and has a distal end or head configured to engage the proximal end of the constricted occluding device and urge the occluding device out a discharge port in the distal end of the catheter. The pusher element can be configured so that the proximal end thereof will extend out of the distal end of the delivery catheter when deploying the occluding device to facilitate the manipulation of the pusher element. Because the occluding device can be capable of being compressed to a very low profile, the delivery catheter may be restricted to very small transverse dimensions. Suitable delivery catheters may have an inner diameter of about 0.008 to about 0.08 inch (0.2-2.00 mm), preferably about 0.015 to about 0.025 inch (0.4-0.6 mm). The smaller diameter delivery catheters reduce the pain and discomfort of delivering the occluding device to the intracorporeal location within the patient. Moreover, the small diameter catheter greatly increases the locations which these occluding devices can be deployed.

In an embodiment, the denuding elements preferably have a ribbon shape, and are secured (e.g., crimped by platinum rings) to the central portion of the occlusion device. There can be a plurality of ribbons, disposed circumferentially around the central portion of the occlusion device. The ribbon can comprise one ribbon segment, secured at both ends of the occlusion device, or can comprise a plurality of ribbon segments, secured at different locations of the central portion. Other denuding elements can also be used, such as spider-line segments.

The denuding element can include an atraumatic (non-traumatic) design at some of the contact points with the lumen's wall. The non-traumatic contacts may be ball-shaped, contoured or ripple ribbon or wire, or a pad. Examples of contoured ribbon include coils, sharp bends, large radius bends. The non-traumatic ends may also be textured in encourage tissue growth. However, in other embodiments, the denuding element can include a portion that is designed to contact and harm at least a few layers of cells along the lumen's wall.

In an embodiment, the present invention discloses a catheter rotation mechanism within a device handle, and this mechanism can include a converter to convert a portion of a linear motion into a rotational motion. The present mechanism can give the deploying catheter the ability to be rotated a given degree to assist in placement and/or tissue disruption through a rotating denuding feature. For a rotational denuding action, the catheter might need to be rotated, for example, through a device handle, and such rotational action is translated into the rotational motion of the denuding element of the occlusion device. With the present converter, manual rotation of the handle can be avoided, and the action of releasing the occlusion device and rotating the denuding elements can be accomplished by a single linear motion or optionally by a combination of a linear motion at the handle and a rotational motion at the handle.

In an embodiment, a delivery system is inserted to a body lumen to the proper location for deployment of an occlusion device. The occlusion device is then partially released, rotated and fully released at the desired location, all through a single linear motion of a handle of the delivery system. A portion of the linear motion of the handle is converted to a rotational motion through a converter attached to the catheter, allowing the catheter to rotate by a linear motion of the handle. For example, a first portion of the linear motion of the handle is preserved, linearly releasing the occlusion device partially from the catheter to the body lumen. A second portion of the linear motion of the handle is converted to a rotational motion, rotating the catheter, which in turn rotates the denuding elements of the occlusion device. The occlusion device can be fully deployed at the end of this motion, and the catheter is retracted. Alternatively, a third portion of the linear motion of the handle can be included, preserving the linear motion to linearly releasing the occlusion device, either completely or still partially. Further linear motions of the handle can also be included, further rotating and releasing the occlusion device until complete deployment.

The delivery system embodying features of an embodiment of the invention has at least a converter coupled to a catheter for converting a linear motion to a rotational motion. The converter component can be a part of the catheter, or can be a separate component attached to the catheter. The converter component can also coupled to a device handle, to allow, for example, an operator to perform a linear motion at the handle, and which translates into a composite linear/rotational motion of the occlusion device. The converter component can be a part of the handle, or can be a separate component attached to the handle.

In an embodiment, the converter component comprises a cam path in a main body insert which includes straight and rotational sections. A piston, which is a mating component to the main body insert, contains a follower which rides the cam path to control the actual movement of the catheter. Thus the piston, which is driven by a linear drive mechanism (for example, a manually operation or a linear motor actuation) is converted to linear and rotational motions during the actuation of the mechanism, which then can release and rotate the occlusion device.

In an embodiment, the present invention discloses an anti-migration feature for an occlusion device, comprising an expanded anchor to eliminate migration during deployment. In an aspect, the anchor is designed to allow free spinning of the occlusion device, for example, to provide optimum performance for rotating denuding elements. The anchors can be disposed on either or both proximal and distal ends of the occlusion device to eliminate migration in different directions. The anchor preferably comprises an expansive element which then is expanded after releasing to a body lumen.

In an embodiment, a delivery system is inserted to a body lumen to the proper location for deployment an occlusion device. The occlusion device is then partially released, including an anchor to attach the occlusion device to the body lumen, and at least a denuding element to provide denuding action of the body lumen. The anchor and the denuding element comprise expansive elements, which expand after being released from the delivery system (e.g., catheter). The denuding element is then rotated to denude the body lumen, and the delivery system can be retracted. Alternatively, the occlusion device can be further released, for example, releasing the rest of the denuding element and/or a second anchor before retracting the delivery system. The anchors and the denuding element (and/or other expansive elements of the occlusion device) have an independent rotating property, which allows one portion of the body of the occlusion device (a denuding element) to rotate without disturbing the anchor's action which remains anchored and does not rotate.

The occlusion device in one embodiment has at least an anchor coupled to either or both proximal end and distal end of the occlusion device. The anchor is also rotational decoupled from the main body of the occlusion device, allowing the main body (which can include a denuding element) to be freely rotated without moving the anchor. In an aspect, the anchor comprises expansive elements, folded down to be loaded into a catheter, and flipped out or radically expanded when deployed. The anchor can comprise ribbon coils or straight formed ends acting as spikes to hook to the tissue of the body lumen. One end of the anchor can comprise a metal ring crimped on the outboard side. The body of the occlusion device can comprise at least one ribbon denuding element and other expansive element such as core brush and stem wire.

At least certain embodiments of the invention can provide numerous advantages over the art. The configuration of this invention provides for an occluding device that provides a denuding feature, for example, through rotation of the occlusion device. The configuration of the present invention also provides ease of deployment, for example, through a rotational converter mechanism to allow a linear deployment performing both releasing and rotating denuding actions. In addition, the configuration of the present invention also provides anti-migration features, preventing a rotatable occlusion device from migrating in any direction during deployment because of a stationary anchor that does not rotate. It provides for an expandable device that, once expanded and placed, may be very stationary and stable. It is highly efficient in its configuration, and otherwise very adaptable in ways that will be clear to one of skill in the art in view of the drawings and detailed description contained herein.

### BRIEF DESCRIPTION OF THE DRAWINGS

The present invention is illustrated by way of example and not limitation in the figures of the accompanying drawings in which like references indicate similar elements.

FIGS. 1A-1D illustrate an exemplary sequence for the deployment of an occlusion device having denuding features according to embodiments of the present invention.

FIG. 2 illustrates various exemplary denuding sequences according to embodiments of the present invention.

FIG. 3 illustrates an exemplary sequence of the deployment of an occlusion device having denuding features.

FIGS. 4A-4C illustrate various ribbon configurations of an occlusion device.

FIGS. 5A, 5B, 6A and 6B illustrate various configurations and denuding sequences of multiple ribbons disposed along the central portion of the occlusion device.

FIG. 7 illustrates an exemplary configuration and denuding sequence of multiple spider-like elements disposed along the central portion of the occlusion device.

FIG. 8A is an elevational view, partially in sectional view, of a delivery catheter illustrating the advancement of an occluding device embodying features of the invention. The occluding device shown in FIG. 8A is a spider device, but in other embodiments according to the invention, the device can be different than a spider device and can include a denuding element or other aspects and embodiments described herein.

FIGS. 8B and 8C are transverse cross-sectional views of the delivery catheter and guide wire shown in FIG. 8A taken along the lines 6-6 and 7-7 respectively.

FIG. 8D is an elevational view of an over-the-wire type delivery catheter.

FIG. 8E is a transverse cross-section of the over-the-wire delivery catheter shown in FIG. 8D, taken along the lines 9-9.

FIG. 8F is an elevational view, partially in section, of the distal section of the over-the-wire delivery catheter shown in FIG. 8D illustrating the advancement of an occluding device embodying features of the invention within the inner lumen of the delivery catheter by a pusher element.

FIG. 8G is an elevational view, partially in section, of an over-the-wire delivery catheter with a combined guide wire-pusher element advancing an occluding member embodying features of the invention through the inner lumen of the catheter. The occluding device shown in FIGS. 8F and 8G are spider devices, but in other embodiments according to the invention, the device can be any one of the occluding devices described or shown in this disclosure.

FIG. 8H is a transverse cross-sectional view of the delivery catheter shown in FIG. 8I taken along the lines 12-12.

FIGS. 9A-9B illustrate an exemplary converter comprising a cam path with straight and rotational sections.

FIG. 10 illustrates a multiview design of an exemplary molded component of the converter.

FIG. 11 illustrates an exemplary converter with cam path on the handle shaft.

FIGS. 12A-12E illustrates various cam paths for the movement transfer for the length of the occlusion device.

FIG. 13 illustrates an exemplary converter having a rack-and-pinion mechanism.

FIG. 14 illustrates an exemplary converter having a spring-loaded handle.

FIG. 15 illustrates an exemplary sequence of the deployment of an occlusion device having a converter feature.

FIG. 16 illustrates an exemplary sequence of the deployment of an occlusion device having anti-migration features.

FIGS. 17A and 17B illustrate exemplary occlusion devices having an anti-migration feature represented by freely-rotating anchors at the ends of the occlusion device.

FIG. 18A illustrates another exemplary occlusion device with coiled ribbon as anchors.

FIG. 18B illustrates an occlusion device in the deployment form, with the anchor hooking into the tubal tissue.

FIG. 19 illustrates another exemplary occlusion device with straight ribbon as anchors.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” means that a particular feature, structure, or characteristic described in conjunction with the embodiment can be included in at least one embodiment of the invention. The appearances of the phrase “in one embodiment” in various places in the specification do not necessarily all refer to the same embodiment. Although the processes are described below in terms of some sequential operations, it should be appreciated that some of the operations described may be performed in a different order. Moreover, some operations may be performed in parallel rather than sequentially.

The present invention provides devices, systems and methods for the occlusion of various body lumens, preferably including, in at least certain embodiments, enhancing the ingrowth of tissue and particularly endothelial tissue surrounding the occlusion device. In the various aspects of occluding body passageways, particularly useful and benefits for this invention are methods and systems for the delivery of occlusive devices to fallopian tube, vas deferens, bronchus, blood vessel or other anatomical passageway or lumen.

In an embodiment, the present invention provides methods and devices that include a denuding feature having a rotational mechanism within the body lumen to encourage tissue ingrowth to enhance the occlusion of the body lumen by the occlusive device. In accordance with the invention there is provided an implantable occlusion of the foregoing character that comprises rotating a denuding feature of the occlusion device within a body lumen during deployment. In an aspect, the present occlusion device comprises a denuding element which can be an expandable intraluminal member which is disposable in a first constricted configuration wherein it is sufficiently compact to be advanced into the body lumen and subsequently expandable to a second configuration within the body lumen. After being expanded, the denuding element is rotated, for example a full rotation, for denuding the lumen's walls before the occlusion device is released from a delivery catheter.

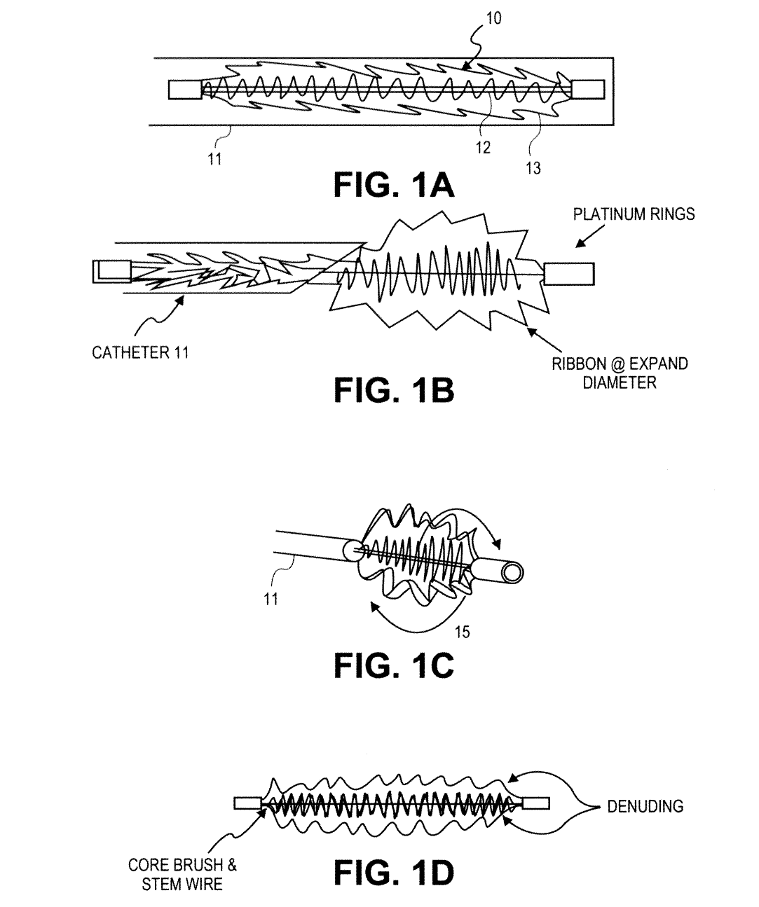

FIGS. 1A-1F illustrate an exemplary sequence for the deployment of an occlusion device having denuding features according to embodiments of the present invention. In FIG. 1A, the occlusion device 10 is in a constricted configuration within a delivery system, such as a catheter 11, which is then inserted into a body lumen (not shown) such as a fallopian tube. The occlusion device as shown comprises a bottle brush configuration 12 for blocking the lumen passageway, and a denuding feature 13 shown in the form of ribbon, crimped at both end by rings such as platinum rings. A typical brush element has a plurality of members with non-traumatic ends radiating from a central location. The members may be interwoven or knotted at the central location. The bottle brush feature and the ribbon shown are simply an exemplary configuration of the present occlusion device having denuding features. Other configurations can also be employed. Further, for simplicity, other elements to the present inventive idea are not included, for example, components of the occlusion device such as extension members extended past the opposing occlusion device, drug delivery component, Dacron or polyester fibers to act as a tissue ingrown promoting agent, or components of the delivery system such as a pusher or a retracter to release the occlusion device to the body lumen.

After reaching a proper location for deploying the occlusion device, the occlusion device 10 is partially released from the catheter 11, by either a retraction of the catheter sheath or a pushing action on the occlusion device or by both retraction and pushing action. FIG. 1B illustrates the occlusion device 10 in a partially released configuration. The bottle brush and the ribbon are expanded to an expanded configuration, where at least the ribbon is contacting the walls of the body lumen. The partially expanded configuration is preferably adequately contacted the lumen's walls, to provide optimum conditions for the denuding features.

After a certain delay, for example, to ensure that the expanded configuration is completed, in FIG. 1C, the occlusion device is rotated 15, rotating the ribbon and denuding the contacted lumen's walls. The rotation action can be accomplished by rotating the catheter 11, which in turn rotates the occlusion device 10. In this case, the remaining of the occlusion device 10 should have enough friction against the catheter, to ensure that the catheter and the occlusion device can rotate together as a unit. Special features can also be implemented to improve rotational motion transfer, such as linear ridges in the catheter to allow ease of linear release of the occlusion device but not permitting free rotation of the occlusion device within the catheter.

After completing the denuding rotation, the occlusion device is deployed the rest of the way and the catheter is removed. In FIG. 1D, the catheter stops rotating, and the occlusion device 10 is completely released from the catheter 11, either by a retraction of the catheter sheath or a forward movement of the occlusion device 10 (through a pushing action of a pusher that has a distal end that pushes a proximal end of the occlusion device).

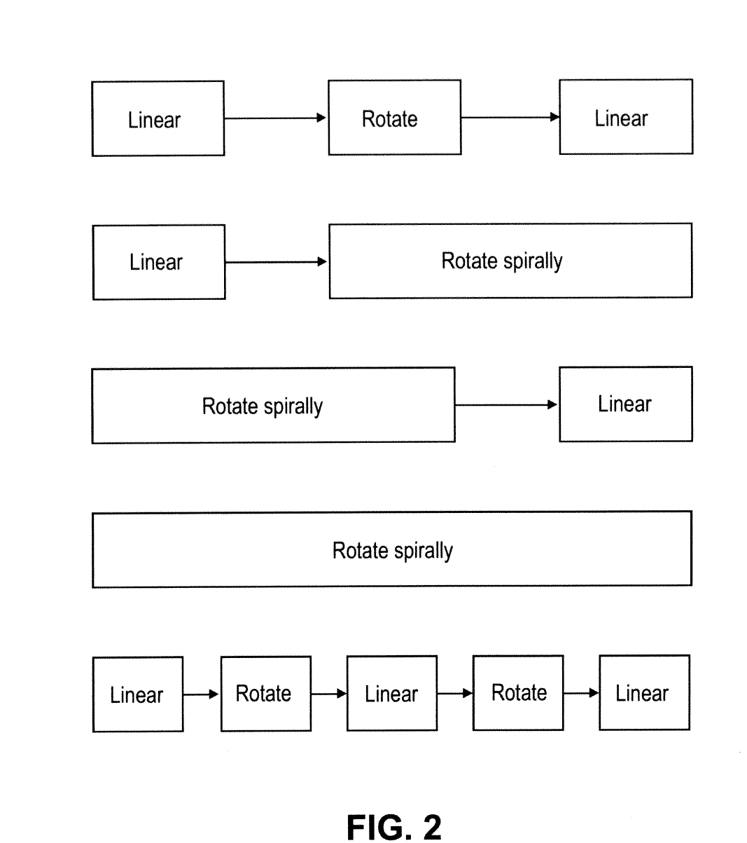

In this exemplary sequence, the denuding action is accomplished by a rotating motion, sandwiched between two linear releasing motions. Other denuding sequences can be used, such as a spirally rotating motion, which can replace a combination of rotating and linear motions. Combinations of motions, such as multiple rotating motions, for example, inserted between linear motions, can also be used. Alternatively, back-and-forth motion can be used, such as rotating back-and-forth or linearly moving back-and-forth longitudinally within the lumen. FIG. 2 illustrates various exemplary denuding sequences according to embodiments of the present invention.

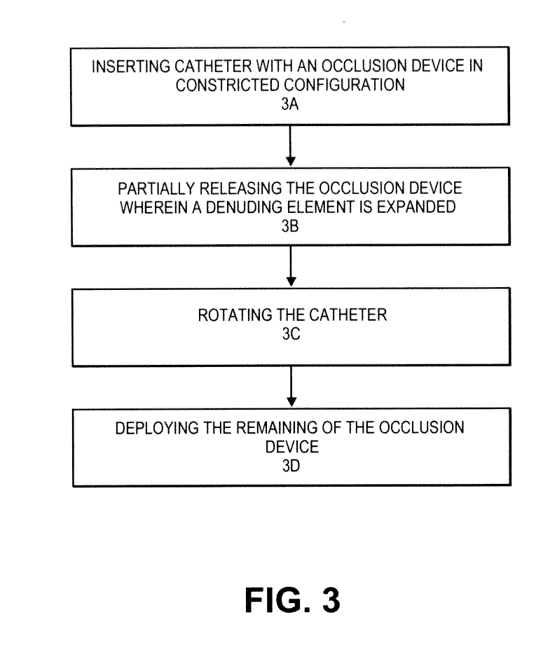

FIG. 3 illustrates an exemplary sequence of the deployment of an occlusion device having denuding features. After preparing a delivery system, such as a catheter with an occlusion device positioned in a constricted configuration (e.g., a radically restrained configuration) in the catheter, the catheter is inserted into a body lumen (e.g. a fallopian tube) (operation 3A). At the appropriate location, the occlusion device is partially released (operation 3B), for example, by retracting the catheter or by pushing the occlusion device forward. The release of the occlusion device can be accomplished by a linear motion or a rotation motion (e.g., spiral motion). In the partially released configuration, the denuding element can be either partially or fully expanded. For example, if the denuding element is a ribbon disposed along the whole length of the occlusion device, only the exposed section of the ribbon is expanded. If the denuding element comprises spider-like segments, it can be fully exposed to the lumen and thus fully expanded. In operation 3C, the expanded denuding element denudes the lumen, for example, by rotating the catheter. The denuding of the lumen can injure the lumen enough to encourage tissue ingrowth without puncturing the lumen's walls; in practice, this can involve removing several layers of cells from the inner wall of the lumen. The amount of rotation can be changed, for example, one full turn, or more or less and one full turn. Other actions can be used to denude the lumen, such as spirally rotating, oscillating rotating or back-and-forth motion. After completing denuding, in operation 3D, the catheter releases the occlusion device, and the catheter is removed.

An innovative feature of the present invention is the inclusion of a rotational denuding feature or action within a deployment action of an occlusion device. During the deployment, the lumen is additionally denuded to provide stimulation of tissue ingrowth. In an aspect, as illustrated above, the denuding action is accomplished by a rotation action, in addition to the linear motions of releasing the occlusion device. Alternatively, other combinations of action can be used, for example, spirally motion to replace a combination of rotating and linear motion, multiple rotation actions to optimize the denuding action, or linearly or rotatingly oscillation.

In an embodiment, the present invention discloses occlusion devices having denuding features. Because tissue healing and the inflammatory reaction to tissue injury involve cell division and cell growth, it is advantageous in some circumstances to irritate, scrape or “injure” the tissue of a lumen wall surrounding an occlusive device of the invention. For example, it may denude the fallopian tube or vessel wall or otherwise irritate the tissue in the region where the occlusive device is placed. This may be accomplished with a denuding fiber bundle on the occlusive device.

The denuding element may be configured, constructed or contain materials that support or facilitate tissue ingrowth. As used herein, the term tissue ingrowth includes but is not limited to cell multiplication and/or or growth resulting in tissue formation into, onto, or surrounding a particular region and/or into, onto or surrounding an obstructive device. This may be epithelization, scar formation, or other cell growth or multiplication. For example, the denuding element may incorporate materials that promote epithelialization, endothelialization, granulation or other proliferative or tissue growth response within the body to create a more effective occlusion of the passageway or to result in a more secure attachment of the occlusion device to the walls of the body lumen. For instance, polyester fibers may be attached to the denuding element such that tissue ingrowth into and around the device will form a plug and thereby occlude the lumen in which the device is implanted. Such as fibers, or other tissue ingrowth promoting agents, can also be attached to other parts of the occlusion device.

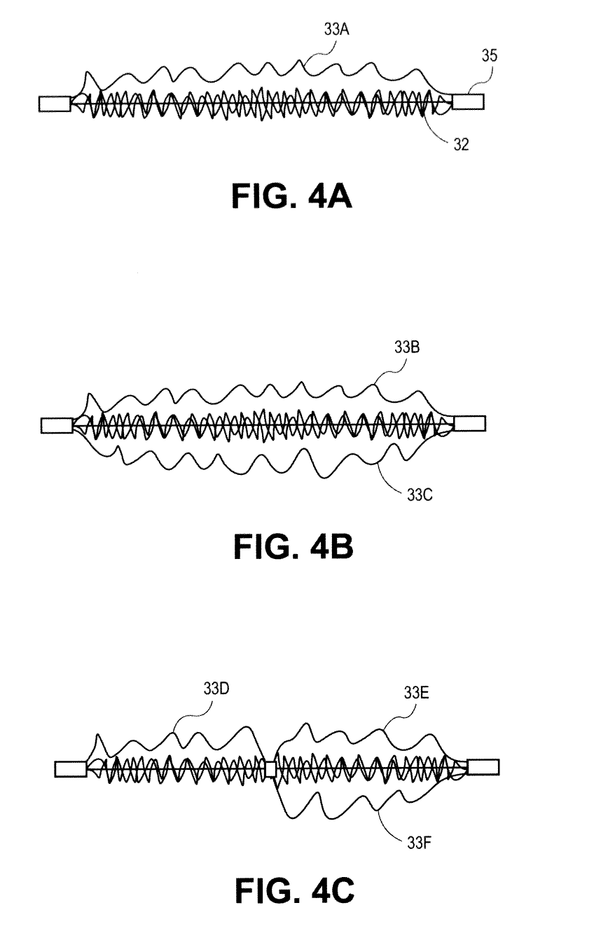

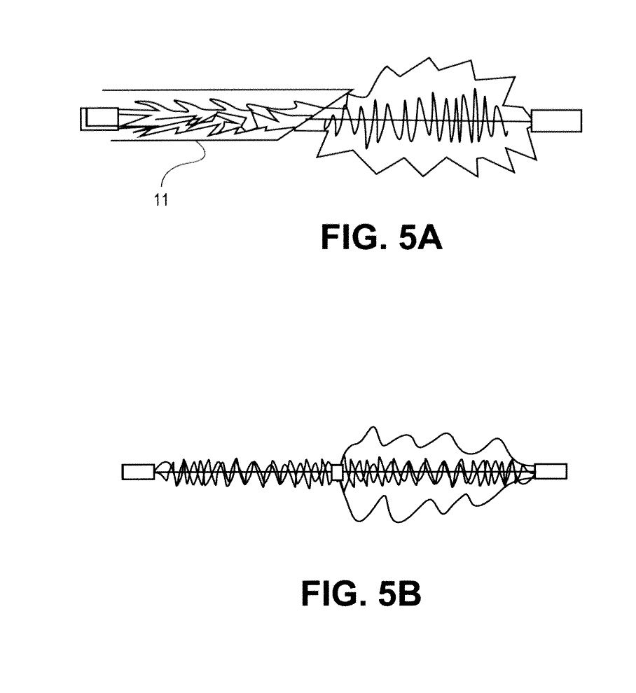

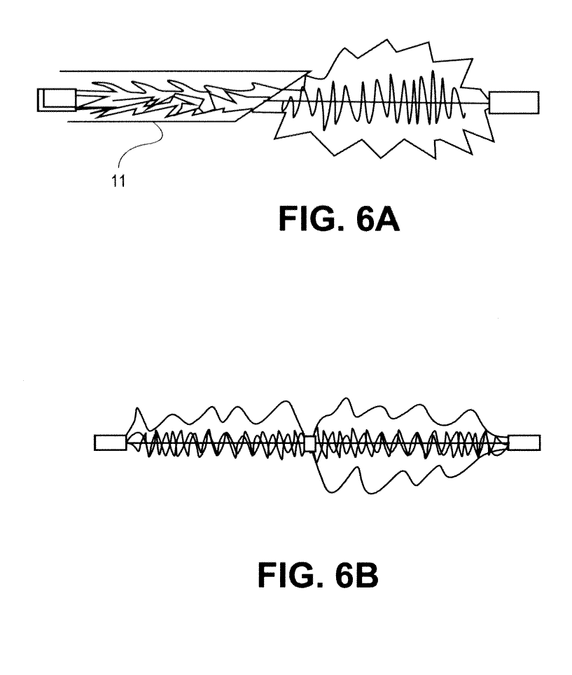

An exemplary denuding feature according to embodiments of the present invention comprises at least a ribbon, or multiple ribbons in a circumferential configuration, disposed along the longitudinal direction of the occlusion device and secured to a central portion of the occlusion device. The ribbons can have flat or circular shape, with contacts such as ripple or twisted designs. The contacts are formed such that they engage a body lumen with a large amount of dragging along the surface area of the lumen wall, without completely piercing the lumen wall. There can also be one or multiple ribbons disposed along the longitudinal direction. FIGS. 4A-4C illustrate various ribbon configurations, including one ribbon 33A crimped to a central portion by a platinum ring (FIG. 4A), two ribbons 33B and 33C (FIG. 4B), three ribbons with one 33D in one section and two 33E and 33F in another section (FIG. 4C). Other features such as a core brush and stem wire 342 can be included. FIGS. 5 and 6 illustrate various configurations of multiple ribbons disposed along the central portion of the occlusion device.

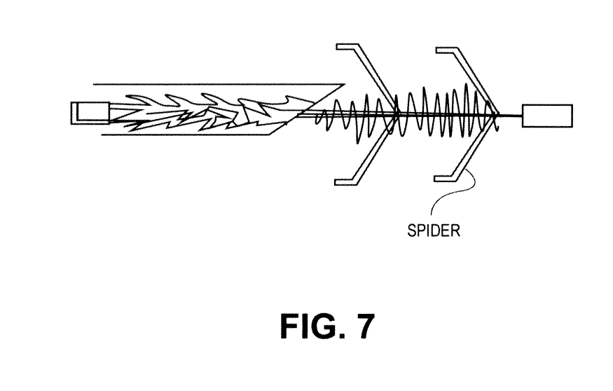

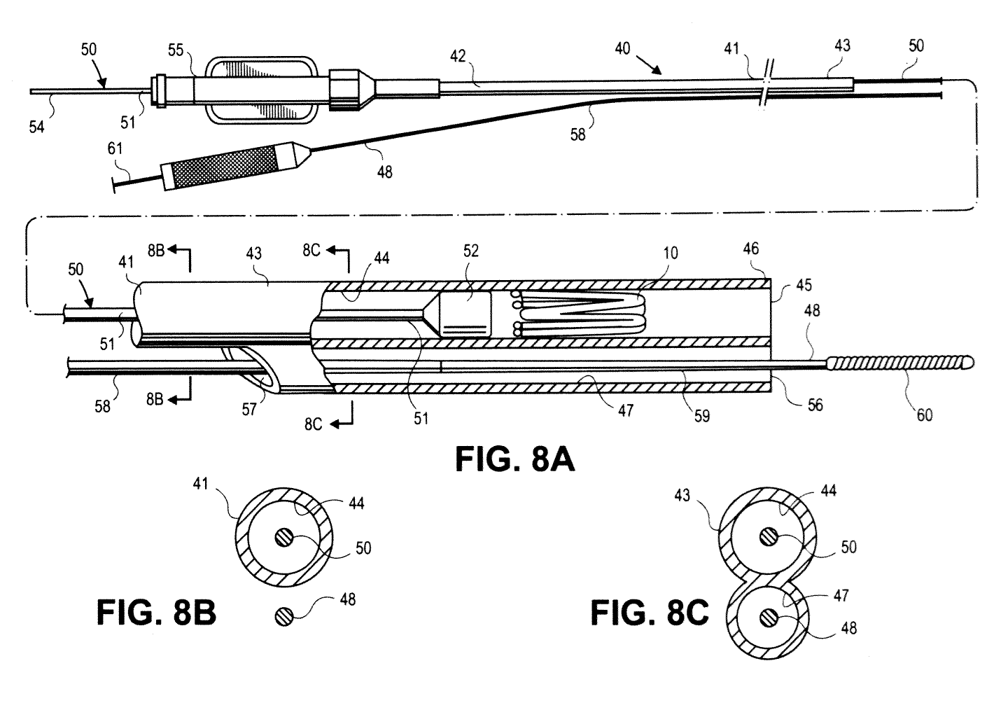

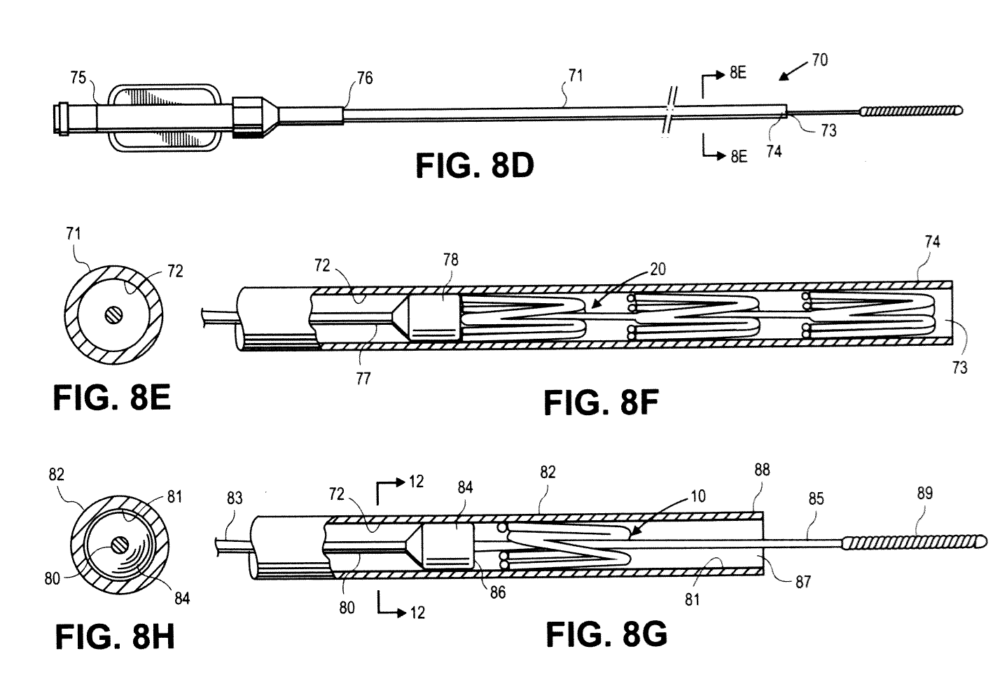

The present invention is not limited to a denuding feature that uses a ribbon configuration. Other expansive elements can be employed as a denuding feature, for example, spider-like expansive segments (such as those shown in FIG. 8A or 8F or 8G) or chemical denuding features can be used with or without mechanical denuding. At lease in certain embodiments, denuding elements are able to fully expand to contact the lumen's wall after being in a constricted configuration in the catheter. FIG. 7 illustrates an exemplary configuration and denuding sequence of multiple spider-like elements disposed along the central portion of the occlusion device. The spider element has a plurality of expansive elements which radiate from a central location, which need not be the geometric center of the device. The center section of the device can include an inner coil, which may be hollow to allow installment by means of a guidewire.

The occluding members embodying features of the invention may be formed at least in part of superelastic NiTi alloy with an austenite to martensite transition temperature less than 40° C., preferably less than 25° C. The occlusion device formed at least in part of superelastic NiTi alloy may have the austenite transformed to martensite by reducing the temperature of the device to below the transformation temperature and then constricting the occluding device to facilitate entry into the inner lumen of the delivery catheter in the martensite phase. The mechanical constriction of the occluding device within the delivery catheter maintains the occluding device in the martensite phase. Alternatively, the device may be mechanically compressed to stress-induce the austenite to martensite transformation. When the NiTi devices are released from the delivery catheter, the NiTi alloy transforms from the martensite phase to the more stable, higher strength austenite phase.

Additionally, the occluding devices embodying features of the invention may be formed at least in part of other high strength biocompatible materials such as MP35N alloy, stainless steel, and high strength biocompatible polymeric materials or combinations thereof may be suitable. Biocompatible polymeric materials such as polyethylene terephthalate (PET) Hytrel or a polyamide such as Nylon 6 or ePTFE, or Silicone may be used. Polymeric materials in combination with metals may be used, such as PET insert molded around NiTi wire.

The occluding devices and placement catheters embodying features of the invention may incorporate coatings which help with insertion and placement of the device. The placement catheters may be coated with a hydrophilic material, such as a PTFE, silicone, or hydrogel, which reduces friction and allows smooth placement of the occluding device into the fallopian tube. The occluding devices may be coated with a hydrophobic material which promotes tissue growth and placement. A hydrophobic coating will become sticky upon placement to help prevent dislodging or movement. The coating in all cases may be fully or partially biodegradable.

An innovative feature of the present invention is the movement of the delivery system to rotate the denuding element of the occlusion device. Thus the occlusion device is preferably not freely rotating within the catheter after partially released to enable motion transfer between the catheter to the occlusion device. Thus the partially released configuration of the occlusion device should not be too far released to decouple to catheter from the occlusion device. Alternatively, a transfer coupling mechanism can be included in the catheter and/or the occlusion device to provide the needed motion transfer. For example, longitudinal ridges can be incorporated to the inside of the catheter or to the outside of the occlusion device to provide adequate friction for rotational transfer and not linear motion transfer. Or spiral ridges along the inner wall of the delivery catheter can be incorporated to allow a linear movement of the occlusion device to spin the occlusion device as it is pushed out of the delivery catheter; these spiral ridges can be configured to mate with rails or ridges on the occlusion device to cause the device to follow the spiral ridges as it is pushed out of the delivery catheter.

The present invention also relates to devices, methods and systems for the occlusion of various passageways of the body including the delivery of therapeutic substances by placement of drugs or drug secreting material on or within such devices. Radiopaque material, such as beryllium sulfate, may also be added to the polymeric material. Furthermore, these materials may include contraceptive drugs (e.g. hormones) which are eluted from the materials to provide contraceptive effect immediately after deployment of the device. The drug(s) may be a spermicidal ingredient or other type of composition.

FIGS. 8A-C show a rapid exchange delivery catheter 40 suitable to deliver an occluding member 10 which can be, rather that the spider device shown in FIG. 8C, any one of the occluding devices described or shown in this disclosure. The delivery catheter 40 has an elongated shaft 41 with a proximal shaft section 42 and a distal shaft section 43. The elongated shaft 41 has a lumen 44 which extends the length of the shaft to the discharge port 45 in the distal end 46 in the distal shaft section 43. The distal shaft section 43 has a second lumen 47 for receiving a guide wire 48 over which the delivery catheter is advanced to the desired intracorporeal location for deploying the occluding device. A pusher element 50 having an elongated shaft 51 has an enlarged head 52 on the distal end thereof to engage an occlusion member 10 slidably disposed within the inner lumen 44. The pusher element 50 is long enough so that the proximal end 54 of the shaft 51 extends out of the proximal end 55 of the catheter 40 when the enlarged head 52 thereof has pushed the occlusion member 10 out the discharge port 45 in the distal end 46 of the catheter into a body lumen. The guide wire 47 is slidably disposed within the short guide wire lumen 47 which may be about 0.5 to about 50 cm, preferably about 10 to about 35 cm in length. A distal guide wire port 56 is provided in the distal end 46 of the catheter 40 and a proximal guide wire port 57 is provided a short distance proximal from the distal guide wire port and a substantial distance from the proximal end 55 of the catheter. The guide wire 47 may be of conventional structure with an elongated shaft 58, a tapered distal shaft section 59 and a shapeable spring tip 60 which enables steering the distal end of the guide wire within the patient's body lumen by torquing the proximal end 61 which is configured to extend out of the patient's body.

When delivering the occlusion device 10 by means of a rapid delivery catheter 40, the guide wire 47 is usually advanced through the patient's vaginal canal and uterine cavity and into the patient's fallopian tube with a hysteroscope. The shaped spring tip 60 on the distal end of the guide wire 47 may be used to guide the distal tip into the patient's fallopian tube. The guide wire 47 is advanced until the spring tip 60 is disposed distal to the desired location for the occluding member 10. The rapid exchange delivery catheter 40 may then be advanced over the guide wire until the distal end of the delivery catheter 40 is in an appropriate position for the delivery of the occluding device within the patient's body lumen. The pusher element 50 is then distally advanced until the enlarged head 52 pushes the occluding device 20 out the discharge port 45 in the distal end 46 of the delivery catheter 40. The occlusion device 10 expands upon deployment from the delivery catheter 40 and then the delivery catheter and guide wire 47 may be removed from the patient.

The movement of the pusher rod and occluding device within the catheter, of course is relative. That is, in one application, the enlarged head may be held stationary in the longitudinal direction, and the catheter with the occluding device therein may be withdrawn, causing the enlarged head to contact and expel the occluding device from within the catheter. Relative to the body lumen, such as the fallopian tube, however, the occluding device does not move. The catheter that is withdrawn and the occlusive device is laid down in the fallopian tube as the catheter is withdrawn. This has the advantage of allowing the occlusive device to be expelled from the catheter lumen into the fallopian tubes so that the occlusive device does not move in a longitudinal direction within the fallopian tube. Since the occlusive device may consist of several spider segments, and since the first one expelled from within the catheter will often expand and engage the wall of the fallopian tube immediately upon release from the confines of the lumen 44, it may be important not to attempt to push the occlusive device in a longitudinal direction once it has begun to attach to the fallopian tube walls.

FIG. 8D-8F depict an over-the-wire type delivery catheter 70 which has an elongated shaft 71, an inner lumen 72, a distal port 73 in the distal end 74 of the shaft and an adapter 75 on the proximal end 76 of the shaft. As shown best in FIG. 8F a pusher rod 77 with enlarged head 78 is slidably disposed within the inner lumen 72. The enlarged head 78 is configured to engage the proximal end of occlusion device 20 which is disposed within the inner lumen 72 in a constricted configuration. Distal movement of the pusher rod 77 advances the occlusion device 20 through the inner lumen and out the distal port 73 in the distal end 74. While the occlusion device shown in FIGS. 8F and 8G are spider devices, it will be understood that the device may alternatively be any one of the occlusion devices described or shown in this disclosure, such as the devices shown in FIGS. 1A-1D, 4A-7, 17A-19.

An alternative delivery system is shown in FIGS. 8G-8H wherein a pusher rod 80 is slidably disposed within an inner lumen 81 of delivery catheter 82. The pusher rod 80 has an elongated shaft 83, an enlarged head 84, and a distal shaft section 85, extending from the front face 86 of the enlarged head 84, is provided with a distal spring tip 86. The pusher rod 80 is in effect a combined pusher rod-guide wire which both guides the delivery system to the desired location and pushes an occlusion device 10 out of the discharge port 87 in the distal end 88 of the delivery catheter 82. In another embodiment, a pusher rod, which is similar to pusher rod 80, engages the proximal end of the occlusion device in order to either push out the device or hole it stationary while the catheter sheath is retracted, but the pusher rod does not include a distal guidewire section. Rather, the occlusion device itself includes a distal guidewire section which is similar to the Essure device from Conceptus, Inc. of Mountain View, Calif. This Essure device, and a delivery system to deliver and deploy the device, are described in U.S. Pat. No. 7,506,650, which patent is hereby incorporated herein by reference.

The devices, systems, and methods of this invention may be used in the occlusion of various body passageways. For example, the occluding devices of the invention may be used to occlude arteries leading to tumors and other undesirable tissue. Additionally, the devices are particularly well-suited for the steerable delivery of small self expanding intravascular devices, including coronary and neurovascular stents. The devices and methods described herein may be placed using visual means, ultrasonic guidance and/or fluoroscopy.

The occluding devices embodying features of the invention may alternatively be placed inside the body without a catheter. Occluding devices may be loaded into a hysteroscope directly and pushed out from an opening in the hysteroscope by a plunger, similar to what is illustrated in FIG. 5F, except a working channel of the hysteroscope being element 74. Additionally a coating may be incorporated on the occluding devices which is hydrophilic to allow ease of insertion.

In an embodiment, the present invention provides methods and devices that simplify the placement of an occlusion device having a denuding element. In some embodiments, the placement of an occlusion device having a denuding element can include a linear motion to release the occlusion device and a rotational motion to denude the body lumen. In accordance with some embodiments of the invention there is provided a delivery system with the ability to be rotated a given degree to assist in placement and tissue disruption. In an embodiment, the present system comprises a motion transfer mechanism, such as a converter, to convert all or only a portion of a linear motion to a rotational motion (including spiral motion).

To provide rotating denuding action, such as the exemplary denuding sequences described herein, the delivery system, such as the handle of the catheter, might need to be rotated, for example, by an operator, in addition to the linear motions to release the occlusion device. Thus the present invention discloses a converter, or a motion transfer mechanism, to allow a linear movement of the delivery system, such as a handle or control on the handle, to perform in both linear placement and rotational denudement of the occlusion device. The converter can be an attachment component to the catheter or to the handle of the delivery system, or an integrated part of the catheter or the handle. In an aspect, the converter comprises a cam/piston action to translate a linear motion of the piston to a combination of linear and rotational actions of the piston. Other converter mechanisms can also be used, such as lead screw, rack-and-pinion drive, or spring-loaded axis etc.

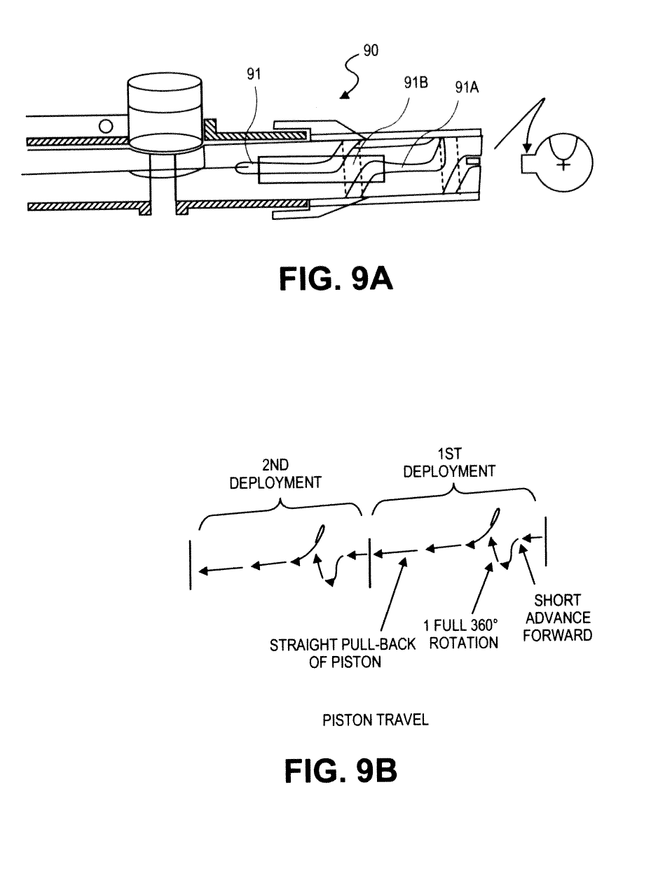

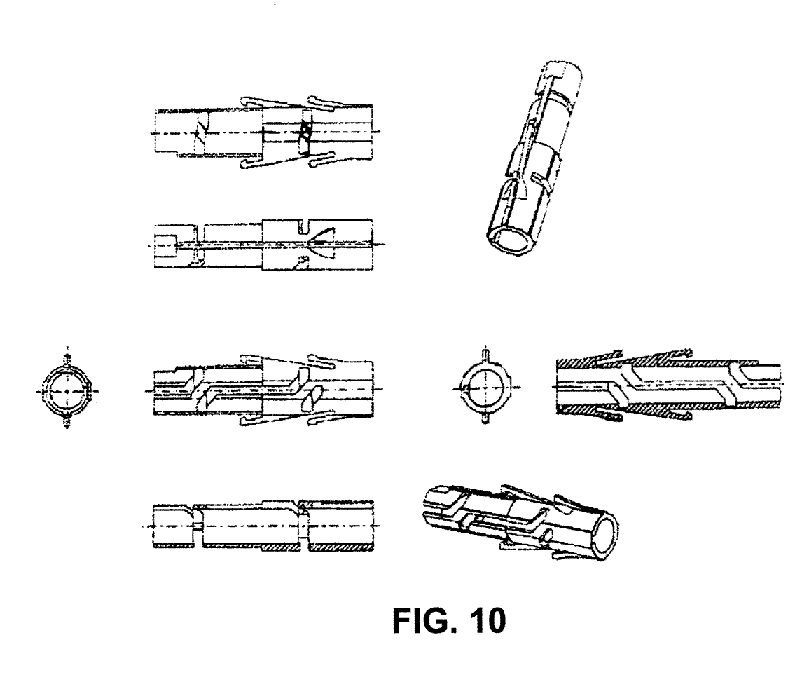

FIG. 9A illustrates an exemplary converter 90 comprising a cam path 91 including straight section 91A and rotational section 91B. The cam path is preferably molded to the inside of the converter for ease of manufacturing and installation. The mating component to the cam path is a piston comprising a follower riding within the cam path to control the movement of the catheter. The piston is driven by a linear drive mechanism, such as an operator manual control or a motor guide, and thus travels along the cam path as shown in FIG. 9B. The movement of the piston is determined by the cam path, and in the exemplary configuration shown in FIGS. 9A and 9B, comprising two deployment movements, each with a short advanced forward, followed by a full 360° rotation, and concluded with a straight pull back of the piston. As the piston gets pulled through the handle of the delivery system, the piston is forced by the flower feature to stay within the spline trajectory of the cam path, traveling straight when the cam path is straight, and turning when the cam path turns. When the piston turns, the catheter should also turn, and this spinning motion will turn the occlusion device, which turns the denuding elements to assist in the removal of lumen cells. FIG. 10 illustrates a multiview design of an exemplary molded component of the converter, having a cam path that comprises two full rotations between three sections of straight path. The converter can be made from metal or plastic, such as ABS or polycarbonate.

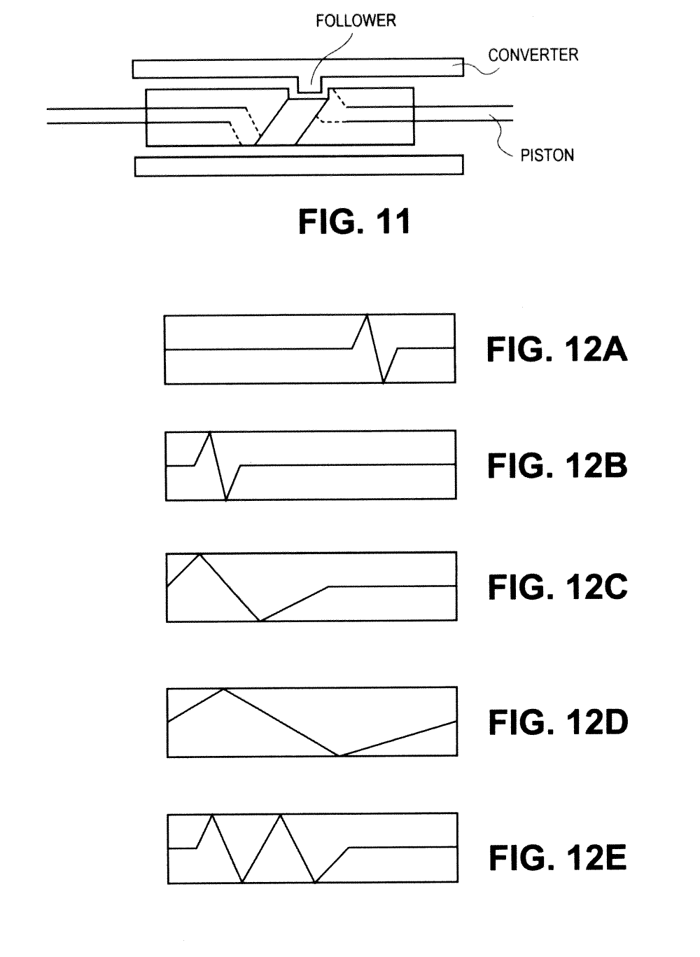

The above earn path configuration illustrates a recess cam path with a protruding follower in the piston. Alternatively, the cam path can comprise a protruding component with the mating component (the follower of the piston) having a recess portion. In other configurations, the cam path (either recess cam path or protruding cam path) can be manufactured on the handle shaft, with the converter comprising a follower disposed on the inside surface (FIG. 11).

FIGS. 12A-12E illustrate various cam paths for the movement transfer for the length of the occlusion device. In FIG. 12A, the piston (which drives the catheter which drives the denuding element of the occlusion device) is retracted, exposing a small portion of the denuding element into the lumen, followed by a full rotation before a large pulling straight back. In FIG. 12B, the piston is retracted, exposing a large portion of the denuding element into the lumen, followed by a full rotation before a small pulling straight back. In FIG. 12C, the piston is retracted, exposing a portion of the denuding element into the lumen, followed by a full spiral rotation. The spiral rotation rotates the occlusion device and at the same time, pulling the occlusion device backward. In FIG. 12D, the piston is rotated spirally the whole length of the occlusion device, pulling the occlusion device backward and rotating at the same time. In FIG. 12E, the piston is retracted, and then rotated two full rotations before pulling straight back. Other cam path configurations can be used to optimize the placement of the occlusion device together with the denuding of the lumen.

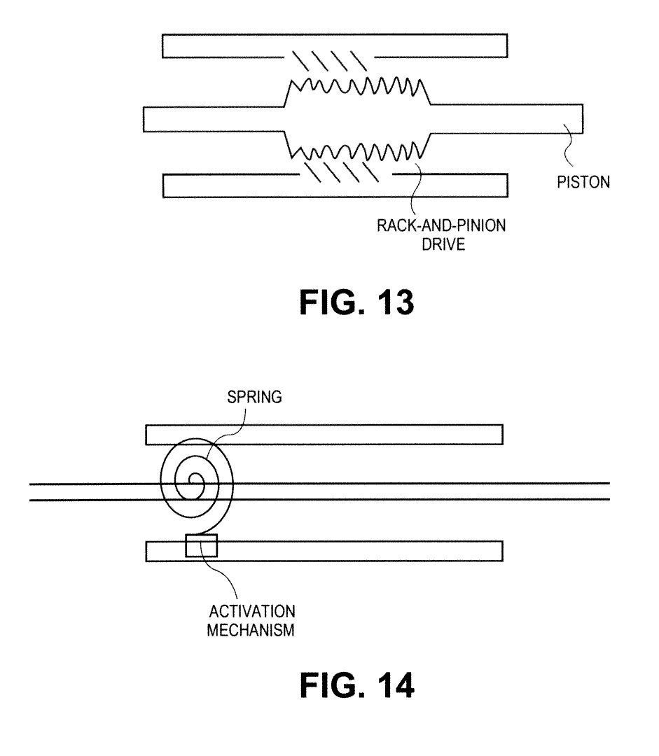

Other converter mechanisms can be used, such as a rack-and-pinion mechanism, transferring a linear motion of the center handle to a rotational movement of the catheter (FIG. 13), or a spring-loaded handle, allowing the handle to spin when reaching a predetermined location in the converter (FIG. 14).

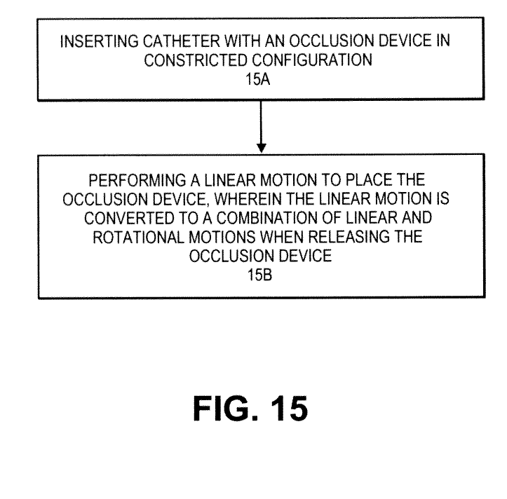

FIG. 15 illustrates an exemplary sequence of the deployment of an occlusion device having a converter feature. After preparing a delivery system, such as a catheter with an occlusion device positioned in a constricted configuration in the catheter, the catheter is inserted to a body lumen (operation 15A). At the appropriate location, a linear motion is performed, for example, on the handle of the delivery system or on a control on the handle, wherein the linear motion is converted by the converter to a combination of linear motion and a rotational motion to release the occlusion device and to denuding the lumen (operation 15B). The combined motion can be a spiral motion, a combination of rotation and straight linear motions, or a combination of spiral and straight linear motions. In certain embodiments, the motion can be only a rotational motion to denude the lumen rather than both linear and rotational motions.

In an embodiment, the present invention provides methods and devices to provide anti-migration features to an occlusion device, especially to an occlusion device having denuding elements. The anti-migration feature can anchor the occlusion device into position after the initial placement to eliminate the chance of migration after implantation.

For example, a device implanted into a body lumen may be subject to forces that would tend to expel the device. This is especially true immediately after placement before the cells of the lumen wall have grown into the occlusive device and helped to secure it to the wall. For example, if an object is placed in the fallopian tubes, the cilia therein may tend to “sweep” the object out of the fallopian tube. Likewise, blood flowing within a vessel may tend to dislodge the device and move it away from the location it was originally placed. This tendency to be expelled or moved is, of course, resisted by the friction of the occlusive device with the walls of the lumen in question. However, if the entire device is unitary and rigid, the force acting to move or expel the device may be sufficient to overcome the friction of the lumen walls with the surface of the device.

Further, the probability of migration can be particular high for occlusion device with denuding elements, since the denuding elements might need rotational movement to denude lumen tissue.

For certain embodiments of a rotatable device, the present invention discloses anchors that allow free spinning of the main occlusion device, for example, to accommodate the rotating denuding action of the occlusion device after anchoring while at the same time preventing migration of the device within the lumen. The anchors are preferably made from expansive element, folded to fit into a catheter, and expanded after deployment to eliminate migration. The anchors are preferable located on either or both proximal and distal ends of the occlusion device to prevent migration in either direction.

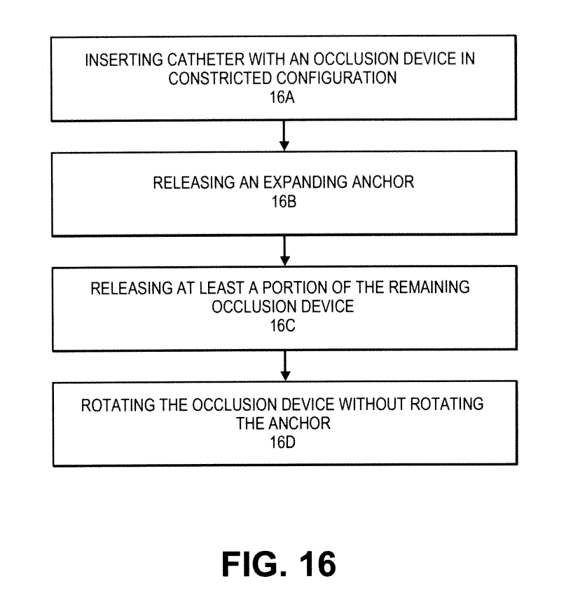

FIG. 16 illustrates an exemplary sequence of the deployment of an occlusion device having anti-migration features. After preparing a delivery system, such as a catheter with an occlusion device positioned in a constricted configuration in the catheter, the catheter is inserted to a body lumen (operation 16A). At the appropriate location, an anchor of the occlusion device is released (operation 16B) to anchor the occlusion device. The occlusion device is further partially released (operation 16C), for example, by further retracting the catheter or by further pushing the occlusion device forward. The release of the occlusion device can be accomplished by a linear motion or a rotation motion (e.g., spiral motion). At the partially released configuration, the denuding element is either partially or fully expanded. In operation 16D, the expanded denuding element denudes the lumen, for example, by rotating the catheter. Since the denuding element can be configured to spin freely relative to the anchor, the denuding action does not interfere with the anchoring action of the occlusion device. After completing the denuding process, the catheter deploys the occlusion device, including an optional second anchor, and the catheter is removed.

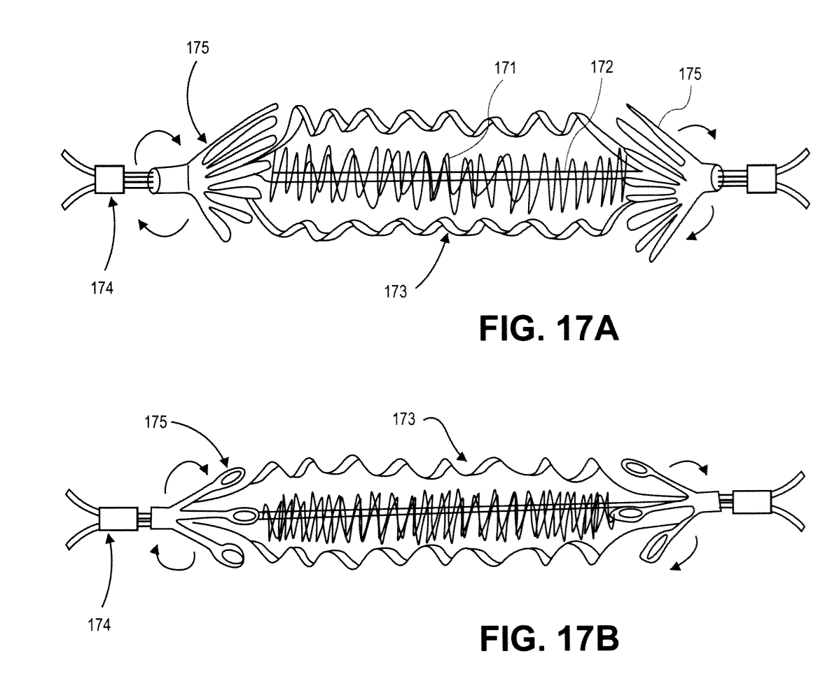

FIGS. 17A and 17B illustrate exemplary occlusion devices having an anti-migration feature represented by freely-rotating anchors at the ends of the occlusion device. The occlusion device shown comprises a core brush 171 surrounding a central wire 172. Two denuding ribbons 173 are also disposed along the longitudinal direction of the occlusion device, for example, to provide denuding action of the lumen. Platinum rings 174 are crimped on the ends of the occlusion device to retain the brush and denuding ribbon. The anti-migration feature is shown as anchors 175 on proximal and distal ends of the occlusion device, allowing for the spinning of the central portion (brush, central wire, and denuding ribbon) independent of the anti-migration features. The central portion can be rotated independently of the anchors 175. The anchors are shown as multiple spikes (FIG. 17A), or multiple spikes with inner eyes (FIG. 17B) radiated from the central portion. The anchors are preferably made from super elastic nitinol, and heat set to the desired final shape.

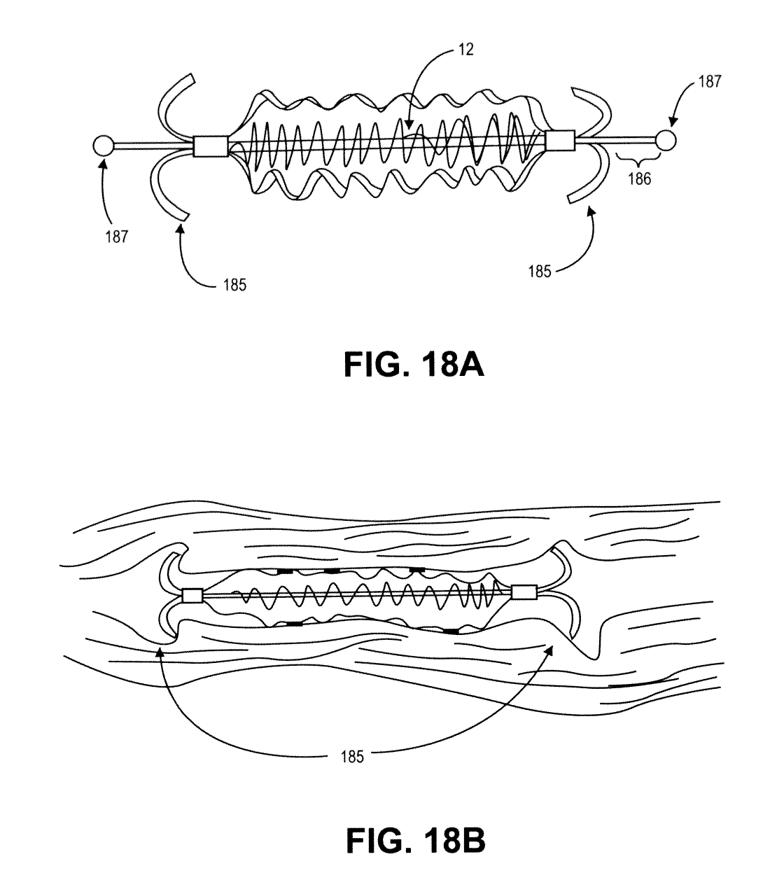

FIG. 18A illustrates another exemplary occlusion device with coiled ribbon as anchors. Multiple ribbons form coils, preferably two coils, at the ends of the occlusion device. The occlusion device also includes an extension portion 186 ended with a welded bulb 187. The extension portion allows the fold down of the anchor coils for easy loading into a catheter for deployment. FIG. 18B illustrates an occlusion device in the deployment form, with the anchor hooking into the tubal tissue.

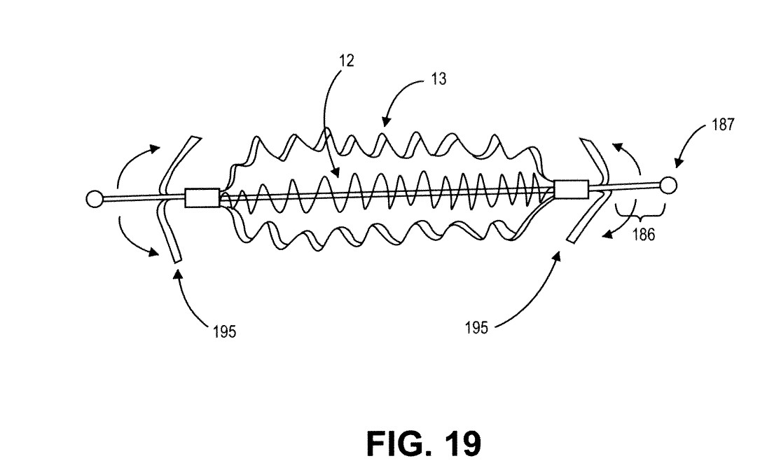

FIG. 19 illustrates another exemplary occlusion device with straight ribbon as anchors. The ribbon is folded down in a constricted configuration when loaded into a catheter, and flipped up when deploy to anchor the occlusion device to the lumen.

While particular forms of the invention have been illustrated and described herein, it will be apparent to those skilled in the art that various modifications and improvements can be made to the invention. Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. It is therefore intended that this invention to be defined by the scope of the appended claims as broadly as the prior art will permit.

Terms such a “element”, “member”, “device”, “sections”, “portion”, “section”, “means”, “steps” and words of similar import when used herein shall not be construed as invoking the provisions of 35 U.S.C. sctn. 112(6) unless the following claims expressly use the term “means” followed by a particular function without specific structure or use of the term “step” followed by a particular function without specific action. All patents and patent applications referred to above are hereby incorporated by reference in their entirety. Accordingly, it is not intended that the invention be limited, except as by the appended claims.

What is claimed is:

1. An occluding member for a patient's lumen having at least one segment with a first constricted configuration and a second expanded configuration, the occluding member comprising:
at least a ribbon disposed along the longitudinal direction of the occluding member and expanded outward when in an expanded configuration,
wherein the ribbon, when in an expanded configuration, denudes the lumen when the ribbon is rotated within the lumen.

2. The member of claim 1 further comprising a bottle brush assembly along the longitudinal direction of the occluding member and inside the ribbon when expanded.

3. The member of claim 1 wherein the bottle brush assembly comprises fibers.

4. The member of claim 1 wherein the ribbon has a rippled formfactor.

5. The member of claim 1 wherein the ribbon is disposed from a proximal end to a distal end of the occluding member.

6. The member of claim 1 wherein the ribbon is formed from a shape memory material.

7. The member of claim 1 wherein the ribbon is formed from at least one of stainless steel, nitinol, and PET (polyethylene terephthalate).

8. The member of claim 1 wherein the ribbon is loaded inside a catheter when in constricted configuration and self-expands after being released from the catheter and wherein the ribbon scrapes the tissue of the patient's lumen when rotated.

9. The member of claim 1 wherein the ribbon denudes the lumen when the occluding member is partially released.

10. A system for occluding a patient's lumen, comprising:
an elongated delivery catheter having a proximal end, a distal end, a port in the distal end, and an inner lumen in fluid communication with the port in the distal end of the catheter; and
an occluding member disposed within the inner lumen of the catheter and configured to expand when released within the patient's lumen,
wherein the occluding member comprises at least a ribbon disposed along the longitudinal direction which expands outwardly when in an expanded configuration and wherein the ribbon is configured to denude the patient's lumen, and
wherein the occluding member, when protruded partly from the inner lumen of the catheter, is still attached to the inner lumen so that when the catheter rotates, the protruded portion of the occluding member is also rotated.

11. The system of claim 10 further comprising a bottle brush assembly along the longitudinal direction of the occluding member and inside the ribbon when expanded.

12. The system of claim 11 wherein the bottle brush assembly comprises fibers which are configured to promote issue ingrowth into the occluding member and wherein the ribbon and the occluding member are self-expansive.

13. A method for denuding a patient's lumen, comprising:
inserting an elongated delivery catheter containing an occluding member, the occluding member comprising a feature configured to denude the patient's lumen when released at least partially from the catheter;
partially releasing the occluding member in the patient's lumen;
rotating the catheter to rotate the partly released occluding member to denude the patient's lumen; and
releasing a remainder of the occluding member in the patient's lumen.

14. The method of claim 13 wherein the occluding member comprises at least a ribbon disposed along the longitudinal direction of the occluding member and which expands outwardly to an expanded configuration.

15. The method of claim 13 wherein the feature configured to denude the patient's lumen comprises at least a ribbon disposed along the longitudinal direction of the occluding member.

16. The method of claim 15 wherein the ribbon is expanded when the occluding member is partially released from the catheter.

17. The method of claim 16 wherein the occluding member is attached to the catheter while the occluding member is rotated to denude the patient's lumen and then the occluding member is completely released.

18. The method of claim 17 wherein the occluding member comprises a bottle brush assembly along the longitudinal direction of the occluding member and inside the ribbon when expanded and wherein the bottle brush assembly comprises fibers which are configured to promote tissue ingrowth into the occluding member and wherein the ribbon and the occluding member are self-expansive.

19. The method of claim 13 wherein the denuding actions occur when the occluding member is partially released.

20. A component to be attached to a delivery catheter, comprising:
a catheter component having a converter mechanism to convert at least a portion of a linear motion along the delivery catheter to a rotational motion of an occluding member disposed on the delivery catheter.

21. The component of claim 20 wherein the converter mechanism further retains a portion of the linear motion to linearly release the occluding member from the catheter.

22. The component of claim 21 wherein the rotational motion of the occluding member serves to denude tissues of a patient's lumen.

23. The component of claim 20 further comprising a handle coupled to the component.

24. A component of claim 20 wherein the inner lumen comprises a cam path to convert a portion of a linear motion of a piston within the inner lumen to a rotational motion of the piston.

25. The component of claim 24 wherein the cam path further retains a portion of the linear motion of the piston to linearly release a occluding member from the catheter.

26. The component of claim 25 wherein the cam path comprises a linear path and a rotational path and wherein the component is molded in one piece.

27. The component of claim 25 wherein the cam path comprises a rotational path sandwiched between two linear paths.

28. The component of claim 25 wherein the cam path comprises a plurality of linear and rotational paths.

29. The component of claim 25 wherein the piston comprises a follower which follows the cam path.

30. A system for occluding a patient's lumen, comprising:
an elongated delivery catheter having a proximal end, a distal end, a port in the distal end, and an inner lumen in fluid communication with the port in the distal end of the catheter;
an occluding member disposed within the inner lumen of the catheter and configured to expand when released within the patient's lumen; and
a converter mechanism configured to convert at least a portion of a linear motion along the inner lumen to a rotational motion of the occluding member.

31. The system of claim 30 wherein a cam path on the converter mechanism uses a portion of the linear motion to release the occluding member from the catheter.

32. The system of claim 30 wherein the rotational motion serves to denude tissues of the patient's lumen and wherein the system further comprises a handle coupled to the component.

33. The system of claim 31 wherein the cam path comprises a linear path and a rotational path.

34. The system of claim 31 wherein the cam path comprises a rotational path sandwiched between two linear paths.

35. The system of claim 31 wherein the piston is coupled to the catheter so that when the piston turns, the catheter also turns and wherein the piston comprises a follower which follows the earn path.

36. A method for delivering an occluding member to a patient's lumen, comprising:
inserting a delivery catheter containing the occluding member, wherein the catheter is attached to a component that converts a linear motion to a rotational motion;
performing a linear motion to release the occluding member in the patient's lumen, wherein the linear motion is converted to at least a rotational motion to rotate the occluding member and a linear motion to release the occluding member.

37. The method of claim 36 wherein a cam path in the delivery catheter retains a portion of the linear motion of the piston to linearly release the occluding member from the catheter.

38. The method of claim 36 wherein the rotational motion serves to denude tissues of the patient's lumen.

39. The method of claim 37 wherein the cam path comprises at least one linear path and at least one rotational path.

40. An occluding member for a patient's lumen, comprising:
a proximal end and a distal end;
a central implant segment disposed between the proximal end and the distal end with a first contracted configuration and a second expanded configuration;
at least an anchor disposed at least one of the proximal end and the distal end, the anchor configured to expand to prevent the occluding member from migrating after placement,
wherein the central implant segment is configured to rotate independently of the anchor.

41. The member of claim 40 further comprising a second anchor disposed at the other end.

42. The member of claim 40 wherein the anchor has a non-traumatic shape.

43. The member of claim 40 wherein the anchor comprises at least one coil.

44. The member of claim 40 wherein the central implant segment comprises an expansive element.

45. An occluding member configured to be placed within a fallopian tube, the occluding member comprising:
a rotating segment;
an anchor segment coupled to the rotating segment, the anchor segment configured to expand and to engage a wall of the fallopian tube, and the rotating segment configured to rotate to denude a portion of the wall of the fallopian tube and wherein the rotating segment is rotatable without rotating the anchor segment.

46. The occluding member as in claim 45, wherein the anchor segment is at a first end of the occluding member and the rotating segment is at a second end of the occluding member.

47. A method for delivering an occluding member to a patient's lumen, comprising:
inserting a delivery catheter containing the occluding member into the patient's lumen;
releasing an anchor of the occluding member and a rotating portion of the occluding member;
rotating the rotating portion of the occluding member in the patient's lumen without rotating the anchor.

48. The method of claim 47 further comprising:
detaching the occluding member from the delivery catheter, and wherein the anchor is configured to expand and to engage a wall of the patient's lumen and the rotating portion is configured to rotate to denude a portion of the wall.

49. The method of claim 48 wherein the occluding member comprises fibers which are configured to promote tissue ingrowth into the occluding member.
